SEQUENCE LISTING (12 sequences)

SEQ ID NO: 1 - protein, 382 aa, homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 2 - protein, 382 aa, rattus norvegicus
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVEMHLKQIEIKKFKYGIEEHGKVKMRGGLLRTYIISILFKSVFEVAFLLIQWYIYGFSLSAVYTCKRDPCPHQVDCFLSRPTEKTIFIIFMLVVSLVSLALNIIELFYVFFKGVKDRVKGRSDPYHATTGPLSPSKDCGSPKYAYFNGCSSPTAPLSPMSPPGYKLVTGDRNNSSCRNYNKQASEQNWANYSAEQNRMGQAGSTISNSHAQPFDFPDDNQNAKKVAAGHELQPLAIVDQRPSSRASSRASSRPRPDDLEI

SEQ ID NO: 3 - protein, 264 aa, homo sapiens
MPRSFLVRKPSDPNRKPNYSELQDSNPEFTFQQPYDQAHLLAAIPPPEILNPTASLPMLIWDSVLAPQAQPIAWASLRLQESPRVAELTSLSDEDSGKGSQPPSPPSPAPSSFSSTSVSSLEAEAYAAFPGLGQVPKQLAQLSEAKDLQARKAFNCKYCNKEYLSLGALKMHIRSHTLPCVCGTCGKAFSRPWLLQGHVRTHTGEKPFSCPHCSRAFADRSNLRAHLQTHSDVKKYQCQACARTFSRMSLLHKHQESGCSGCPR

SEQ ID NO: 4 - protein, 264 aa, rattus norvegicus
MPRSFLVRKPSDPRRKPNYSELQDACVEFTFQQPYDQAHLLAAIPPPEVLNPAASLPTLIWDSLLVPQVQPVAWATLPLRESPRAAELTSLSDEDSGKSSQPPSPPSPAPSSFSSTSASSLEAEAFIAFPGLGQLPKQLARLSVAKDPQSRKAFNCKYCNKEYLSLGALKMHIRSHTLPCVCTTCGKAFSRPWLLQGHVRTHTGEKPFSCSHCNRAFADRSNLRAHLQTHSDVKRYQCQACARTFSRMSLLHKHQESGCSGGPR

SEQ ID NO: 5 - RNA, 1314 nt, homo sapiens
ggcuuuuagcgugaggaaaguaccaaacagcagcggaguuuuaaacuuuaaauagacaggucugagugccugaacuugccuuuucauuuuacuucauccuccaaggaguucaaucacuuggcgugacuucacuacuuuuaagcaaaagaguggugcccaggcaacaugggugacuggagcgccuuaggcaaacuccuugacaagguucaagccuacucaacugcuggagggaagguguggcugucaguacuuuucauuuuccgaauccugcugcuggggacagcgguugagucagccuggggagaugagcagucugccuuucguuguaacacucagcaaccugguugugaaaaugucugcuaugacaagucuuucccaaucucucaugugcgcuucuggguccugcagaucauauuugugucuguacccacacucuuguaccuggcucauguguucuaugugaugcgaaaggaagagaaacugaacaagaaagaggaagaacucaagguugcccaaacugauggugucaauguggacaugcacuugaagcagauugagauaaagaaguucaaguacgguauugaagagcaugguaaggugaaaaugcgagggggguugcugcgaaccuacaucaucaguauccucuucaagucuaucuuugagguggccuucuugcugauccagugguacaucuauggauucagcuugagugcuguuuacacuugcaaaagagaucccugcccacaucagguggacuguuuccucucucgccccacggagaaaaccaucuucaucaucuucaugcuggugguguccuuggugucccuggccuugaauaucauugaacucuucuauguuuucuucaagggcguuaaggaucggguuaagggaaagagcgacccuuaccaugcgaccaguggugcgcugagcccugccaaagacugugggucucaaaaauaugcuuauuucaauggcugcuccucaccaaccgcuccccucucgccuaugucuccuccuggguacaagcugguuacuggcgacagaaacaauucuucuugccgcaauuacaacaagcaagcaagugagcaaaacugggcuaauuacagugcagaacaaaaucgaauggggcaggcgggaagcaccaucucuaacucccaugcacagccuuuugauuuccccgaugauaaccagaauucuaaaaaacuagcugcuggacaugaauuacagccacuagccauuguggaccagcgaccuucaagcagagccagcagucgugccagcagcagaccucggccugaugaccuggagaucuag

SEQ ID NO: 6 - DNA, 21 bp, artificial
  synthetic nucleotide
cagugcacauguaacuaautt

SEQ ID NO: 7 - DNA, 21 bp, artificial
  synthetic nucleotide
auuaguuacaugugcacugtt

SEQ ID NO: 8 - RNA, 21 nt, artificial
  synthetic nucleotide
aacagugcacauguaacuaau

SEQ ID NO: 9 - DNA, 21 bp, artificial
  synthetic nucleotide
gguaagcuucccuggucuatt

SEQ ID NO: 10 - DNA, 21 bp, artificial
  synthetic nucleotide
uagaccagggaagcuuacctt

SEQ ID NO: 11 - RNA, 21 nt, artificial
  synthetic nucleotide
cagguaagcuucccuggucua

SEQ ID NO: 12 - DNA, 32 bp, artificial
  synthetic nucleotide
tgctcaacccagtcaggtgatgcctgaacaaa

(19) United States
(12) Patent Application Publication
(10) Pub. No.: US 2011/0275574 A1
(43) Pub. Date: Nov. 10, 2011

(54) METHOD OF INHIBITING PROLIFERATION OF HEPATIC STELLATE CELLS

(75) Inventors: Lang Zhuo, Singapore (SG); Michelle Chin Chia Lim, Singapore (SG); Gunter Maubach, Singapore (SG)

(73) Assignee: Agency for Science, Technology and Research, Connexis (SG)

(21) Appl. No.: 13/144,526

(22) PCT Filed: Jan. 15, 2010
(86) PCT No.: PCT/SG2010/000009

§ 371 Date: Jul. 14, 2011

Related U.S. Application Data
(60) Provisional application No. 61/193,998, filed on Jan. 16, 2009.

(51) Int. Cl. A61K 38/17; A61K 31/7088; A61P 35/00; A61P 1/16; A61K 31/7105; A61K 31/711; C12N 5/071; A61K 31/713

(52) U.S. Cl. 514/19.3; 435/375; 514/21.2; 514/44 R; 514/44 A

(57) ABSTRACT

There is presently provided a method of inhibiting proliferation of a hepatic stellate cell comprising directly down-regulating the hepatic stellate cell proliferation activity of connexin-43 in the hepatic stellate cell for the treatment of hepatic fibrosis or related disorder. This method primarily involves down regulating connexin-43 with agents such as Snai1, siRNAs, antisense RNA and DNA enzymes directed against the connexin-43 transcript. As a result the proliferation of activated hepatic stellate cells is down regulated.

### CROSS-REFERENCE TO RELATED APPLICATION

This application claims benefit of, and priority from, U.S. provisional patent application No. 61/193,998, filed on Jan. 16, 2009, the contents of which are hereby incorporated herein by reference.

### FIELD OF THE INVENTION

The present invention relates to methods of inhibiting proliferation of a hepatic stellate cell.

### BACKGROUND OF THE INVENTION

The hepatic stellate cells (HSCs) play an important role in the repair process after liver injury by contributing to the accumulation of the extracellular matrix (ECM) proteins. Essentially, HSCs become activated to a proliferative and contractile myofibroblast-like phenotype. This activation process is initiated and sustained by both paracrine and autocrine signaling involving numerous cytokines (Gressner et al., 2007). Paracrine stimulation depends on many different cell types in the liver, for instance the hepatocytes, endothelial cells, platelets and Kupffer cells. These cells secrete different, cytokines like TGF-β1, PDGF, bFGF, and EGF (Friedman, 2008).

HSCs are believed to play a role in the pathogenesis of a number of clinically important conditions such as, for example, hepatic fibrosis, cirrhosis, portal hypertension and liver cancer (Geerts (2004), J. Hepatol. 40(2):331). Hence, HSCs have also become a target for the development of anti-fibrotic therapies (Bataller et al., (2001), Semin Liver Dis. 21(3):437; Bataller et al., (2005), J. Clin Invest. 115(2):209; Friedman (2003), J. Hepatol. 38 Suppl 1:S38).

Activation of HSCs is a dominant event in fibrogenesis. During activation, quiescent vitamin A storing cells are converted into proliferative, fibrogenic, proinflammatory and contractile ‘myofibroblasts’ (Friedman (2003), J. Hepatol. 38 Suppl 1:S38; BataIler et al., (2005), J. Clin Invest. 115(2):209; Cassiman et al. (2002), J. Hepatol. 36(2):200). HSC activation proceeds along a continuum that involves progressive changes in cellular function. In vivo, activated HSCs migrate and accumulate at the sites of tissue repair, secreting large amounts of ECM components and regulating ECM degradation (Cassiman et al. (2002), J. Hepatol. 36(2):200).

### SUMMARY OF THE INVENTION

In one aspect, the present invention provides a method of inhibiting proliferation of a hepatic stellate cell, comprising directly down-regulating the hepatic stellate cell proliferation activity of connexin 43 in the hepatic stellate cell.

The hepatic stellate cell may be in vitro or in vivo, and may be affected by hepatic fibrosis or a hepatic fibrosis related disorder. The hepatic stellate cell may be an activated hepatic stellate cell.

Down-regulating may comprise delivering into the hepatic stellate cell one of the following: Snai1, a nucleic acid molecule encoding Snai1, an siRNA directed against a Cx43 transcript, a nucleic acid molecule encoding an siRNA directed against a Cx43 transcript, an antisense RNA directed against a Cx43 transcript, a nucleic acid molecule encoding an antisense RNA directed against a Cx43 transcript, a DNA enzyme directed against a Cx43 transcript or a nucleic acid molecule encoding a DNA enzyme directed against a Cx43 transcript.

In another aspect, the present invention provides use of an agent that down-regulates the hepatic stellate cell proliferation activity of connexin 43 or a nucleic acid molecule encoding an agent that down-regulates the hepatic stellate cell proliferation activity of connexin 43 for inhibiting proliferation of a hepatic stellate cell, including in the manufacture of a medicament for inhibiting proliferation of a hepatic stellate cell.

In another aspect, the present invention provides use of an agent that down-regulates the hepatic stellate cell proliferation activity of connexin 43 or a nucleic acid molecule encoding an agent that down-regulates the hepatic stellate cell proliferation activity of connexin 43 for treating hepatic fibrosis or a hepatic fibrosis related disorder in a subject, including in the manufacture of a medicament for treating hepatic fibrosis or a hepatic fibrosis related disorder in a subject.

In another aspect, the present invention provides an agent that down-regulates the hepatic stellate cell proliferation activity of connexin 43 or a nucleic acid molecule encoding an agent that down-regulates the hepatic stellate cell proliferation activity of connexin 43 for inhibiting proliferation of a hepatic stellate cell, or for treating hepatic fibrosis or a hepatic fibrosis related disorder in a subject.

The agent may comprise Snai1, an siRNA directed against a Cx43 transcript, an antisense RNA directed against a Cx43 transcript or a DNA enzyme directed against a Cx43 transcript.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

The figures illustrate, by way of example only, embodiments of the present invention, as follows.

FIG. 1. Effect of different concentrations of hTGF-β1 on Cx43 mRNA and protein expression. HSC-2 and 10 days in vitro activated primary. HSCs (pHSCs) were treated with 1 and 10 ng/ml hTGF-β1 for 10 and 24 hours for mRNA and protein analysis, respectively. (A) The mRNA expression of Cx43 was obtained by quantitative real-time PCR and the data were analyzed as fold change relative to the control. The data represent the mean±SD of three independent experiments (*P<0.05, **P<0.005). (B) Ten micrograms total protein was applied for Cx43 analysis in Western blot. The β-actin expression was shown as the loading control. A representative blot for each cell source is shown. (C) The band intensities were estimated using ImageJ and normalized against β-actin. The data represent the average±SD of two to three independent experiments (*P<0.05, **P<0.005).

FIG. 2. hTGF-β1 increased the phosphorylation of Cx43 in HSC-2. (A) Following 6 hours treatment with 10 ng/ml hTGF-β1, the cells were harvested for total cell lysate. Ten and forty micrograms total protein was applied for Cx43 and pCx43 analysis in Western blot, respectively. A representative blot for one of three independent experiments is shown. The band intensities were estimated using the ImageJ software. The expression of pCx43 was normalized against Cx43. The data represent the average±SD of three independent experiments (**P<0.005). (B) HSC-2 cells were treated with 5 μM PKC inhibitor (BIM I) 30 min before treatment with 10 ng/ml hTGF-β1, or with BIM I only. Ten micrograms total protein was applied for pCx43 analysis in Western blot. A representative blot for one of two independent experiments is shown. The band intensities were estimated using the ImageJ software. (C) HSC-2 cells on cover slips were stained with Cx43 and pCx43 S368 antibodies for immunofluorescence. Cx43 was mostly localized in the membrane (top), while pCx43 S368 showed some membrane and for the most part cytosolic staining (bottom right). The bottom left image shows the cells stained with the secondary antibody alone (control).

FIG. 3. FRAP analysis of gap junction intercellular communication in HSC-2. Cells were incubated with 5,6-carboxyfluorescein diacetate in culture medium without phenol red for 30 minutes. After rinsing, cells were analyzed at room temperature. Left panel: Image of the target cell before bleaching (arrow). Middle panel: Image of the target cell after bleaching. Right panel: Image of the target cell after 15 minutes of fluorescence recovery. (A) No recovery of fluorescence in an isolated cell was observed. (B) A contacting cell was examined. Recovery of fluorescence in the target cell was caused by influx of dye from adjacent cells. (C) A representative experimental curve depicts the gradual increase in fluorescence intensity after bleaching of a contacting cell. The data were fitted to the recovery function to calculate the time constant of recovery (τ). (D) Cells were treated with 10 ng/ml hTGF-β1 alone, 5 μM BIM 1 and 10 ng/ml hTGF-β1 or 40 μM carbenoxolone for six hours before FRAP analysis. The transfer constant (k) was calculated from k=1/τ and normalized by dividing by the number of cells (N) in contact with the target cell. The data represent the average±SD (*P<0.05, **P<0.005).

FIG. 4. Analysis of Cx43 and Snai1 transcript and protein level after hTGF-β1 treatment or Snai1 siRNA transfection. The mRNA expression of Cx43 and Snai1 was obtained by quantitative real-time PCR and analyzed as fold change relative to the control. Protein expression was determined by Western blot. A representative blot for one of three independent experiments is shown. The numbers represent the band intensities normalized against β-actin, which were estimated using the ImageJ software. (A) HSC-2 and 10 days in vitro activated primary HSCs (pHSCs) were treated with 1 and 10 ng/ml hTGF-β1 for 10 hours. Data represent the mean±SD of three independent experiments (*P<0.05, **P<0.005). (B) HSC-2 cells were transfected with Snai1 siRNAs 1 or 3 for 24 hours. There is a decrease in Snai1, and a correlated increase in Cx43 on both the mRNA and protein level. The mRNA data represent the mean±SD of three independent experiments (*P<0.005). (C) HSC-2 cells were treated with 10 ng/ml hTGF-β1. Cells were harvested after 2, 6 and 10 hours for mRNA studies. The mRNA data represent the mean±SD of three independent experiments (*P<0.05, **P<0.005 compared to 0 hour, ANOVA). (D) HSC-2 cells were treated with 10 ng/ml hTGF-β1. Cells were harvested after 10, 24 and 30 hours for Western blot analysis of Snai1 and Cx43. A representative blot for one of two experiments is shown. The numbers represent the band intensities normalized against β-actin, which were estimated using the ImageJ software.

FIG. 5. Binding of Snai1 to the potential Snai1 recognition sequence (CAGGTG) in the rat Cx43 promoter. (A) EMSA was performed using 5 μg nuclear extract of 12 days in vitro activated HSCs. Lane 1: A higher molecular weight band ensuing the binding of Snai1 to the oligonucleotide probe was observed. Lane 2: In the competition reaction using 200-fold excess of unlabeled oligonucleotides, no shift in band was observed. Lane 3: No shift was seen in the absence of nuclear extract in the reaction. Lane 4: The mutated oligonucleotide probe was unable to bind to Snai1 in the nuclear extract. (B) EMSA was performed using 5 μg nuclear extract of HSC-2 treated with 10 ng/ml hTGF-β1 for 2 hours or Snai1 siRNAs for 24 hours. There is an increase in the intensity of the band, corresponding to the Snai1-oligonucleotide complex, of the hTGF-β1-treated HSC-2 in comparison to untreated HSC-2. On the other hand, there is a decrease in the intensity of the gel shift band in the Snai1 siRNAs-transfected cells when compared to the mock-transfected cells. The TATA binding protein (TBP) expression serves as a loading control.

FIG. 6. hTGF-β1 decreased the proliferation of HSC-2 cells as assessed by cell number and expression of the proliferation marker PCNA. Cells were treated with 10 ng/ml hTGF-β1 for 48 hours prior to analysis. (A) Cells were trypsinized and counted as described in Materials and Methods. The data represent the average±SD of three independent experiments (*P<0.05). (B) Ten micrograms total protein was applied for Cx43 and PCNA analysis in Western blot. A representative blot for one of three independent experiments is shown. (C) The band intensities were estimated using ImageJ and normalized against the loading control β-actin. The data represent the average±SD of three independent experiments (*P<0.05, **P<0.005).

FIG. 7. Cx43 siRNA transfection decreased the proliferation of HSC-2 cells as assessed by cell number and expression of the proliferation marker PCNA. Cells were independently transfected with each of two Cx43 siRNAs for 48 hours before analysis. (A) Cx43 mRNA expression was analyzed by quantitative PCR and expressed as fold change relative to the mock-transfected cells. (B) Cells were trypsinized and counted as described in Materials and Methods. All data for (A) and (B) represent the mean±SD of three independent experiments (*P<0.005). (C) Ten and one micrograms total protein was applied for Cx43 and PCNA analysis in Western blot, respectively. A representative blot is shown. (D) The graph is a densitometric analysis of the Western blots. The data represent the mean±SD of three independent experiments (*P<0.05, **P<0.005).

FIG. 8. Effect of Snai1 siRNA on TGF-β1-dependent regulation of cell proliferation. Cells were independently transfected with Sna1 siRNA 1 or 3 and 10 ng/ml hTGF-β1 was added for 48 hours before cell counting and immunoblot analysis of PCNA. (A) Ten micrograms total protein was applied for the study of PCNA expression. β-actin represents the loading control. A representative blot of two experiments is shown. The numbers represent the band intensities normalized against β-actin, which were estimated using the ImageJ software. (B) Cells were trypsinized and counted as described in Materials and Methods. Data represent the mean±SD of three independent experiments (*P<0.005).

### DETAILED DESCRIPTION

The present invention relates to the discovery that connexin 43 (Cx43) affects proliferation rates of the hepatic stellate cells and that down-regulation of Cx43 levels or activity in cells inhibits proliferation of HSCs.

The inventors discovered that TGF-β1 effects the down-regulation of Cx43 in HSCs via transcription repressor protein Snai1. TGF-β1 is one of the most well-studied signaling molecules with diverse effects on HSCs, including regulation of collagen metabolism, contractioh and proliferation (Hellerbrand et al., 1999; Kato et al., 2004; Kharbanda et al., 2004; Saile et al., 1999; Verrecchia and Mauviel, 2007). TGF-β1 is up-regulated during hepatic fibrosis and induces activation of HSCs (Hellerbrand et al., 1999; Kanzler et al., 1999). Work by Saile et al. and Shen et al. indicated that TGF-β1 decreases the proliferation of HSCs by arresting cells at the G₁ phase and simultaneously inhibiting apoptosis (Saile et al., 1999; Shen et al., 2003). Although TGF-β1 is a pro-fibrogenic cytokine with the ‘undesired’ effect of causing the accumulation of ECM proteins in the event of uncontrolled HSCs activation, it may have a positive side in the sense that it inhibits HSC proliferation.

However, since TGF-β1 has diverse effects, including pro-fibrotic effects, the inventors undertook identification of down-stream targets of TGF-β1 in HSCs in order to develop methods of directly inhibiting proliferation rather than via TGF-β1.

The present methods make use of the down-stream anti-proliferation effect of TGF-β1 on HSCs, without the need to administer TGF-β1, thus avoiding any pro-fibrotic effect that TGF-β1 may induce. The identification of connexin 43 as a regulator of HSC proliferation provides a convenient method to inhibit growth of HSCs, particularly in relation to hepatic fibrosis and hepatic fibrosis related disorders.

Connexin 43 is a gap junction protein expressed in HSCs. Gap junctions are microscopic channels formed between adjacent cells that allow for intercellular communication via the exchange of small molecules and ions (cyclic nucleotides, inositol phosphates, Ca²⁺, K⁺). Each gap junction channel is formed by two hemi-channels (connexon) between neighboring cells. The connexon itself consists of an assembly of protein subunits called connexins (Goodenough et al., 1996), of which more than 20 different connexins are known to date (Eyre et al., 2006). In the liver, hepatocytes express connexins 26 and 32 (Cx26, Cx32), whereas nonparenchymal cells (endothelial cells, stellate cells, oval cells, Kupffer cells) express connexin 43 (Cx43) (Gonzalez et al., 2002).

Intercellular communication is an important tool used by cells to effectively regulate concerted responses. HSCs communicate to each other through functional gap junctions composed of Cx43 proteins. The inventors have discovered that exogenous human TGF-β1 (hTGF-β1), a pro-fibrotic stimulus, decreases Cx43 mRNA and protein in an HSC cell line and in primary HSCs. Furthermore, hTGF-β1 increases the phosphorylation of Cx43 at serine 368. These effects lead to a decrease in the gap junction intercellular communication between the HSCs, as shown by gap-FRAP analysis. As well, the binding of zinc finger transcription factor Snai1 from the nuclear extract of HSCs to a Snai1 consensus sequence in the Cx43 promoter was observed. In the same context, Snai1 siRNA transfection results in an up-regulation of Cx43 indicating that TGF-β1 may regulate Cx43 via Snai1. Knockdown of Cx43 by siRNA transfection resulted in a slower proliferation of HSCs. These findings illuminate a newly identified down-stream effect of TGF-β1 in HSCs, namely the regulation of intercellular communication by affecting the expression level and the phosphorylation state of Cx43 through Snai1 signaling, which effect is exploited in the present methods, while circumventing the need for TGF-β1 itself.

The observed effect of TGF-β1 on Cx43 in HSC may differ from the effect of TGF-β1 on Cx43 in various other cell types. For example, work by Pimentel and colleagues showed that exogenous TGF-β1 up-regulated Cx43 expression in cardiac myocytes (Pimentel et al., 2002). Wyatt et al. demonstrated that TGF-β1 had no effect on Cx43 expression per se, but altered instead the phosphorylation status of Cx43 in osteoblast-like cells (Wyatt et al., 2001), and another publication (Neuhaus et al., 2008) showed that TGF-β1 down-regulates Cx43 in detrusor smooth muscle cells. These varied responses in different cell types support the idea of discrete cell-type specific response and the notion that TGF-β1 is a cytokine that exerts pleiotropic effects upon a variety of cell types.

Thus, there is provided a method of inhibiting proliferation of a hepatic stellate cell. The method comprises directly down-regulating the hepatic stellate cell proliferation activity of connexin 43. Down-regulating may include delivering into the HSC an agent that down-regulates the HSC proliferation activity of Cx43 or a nucleic acid that encodes such an agent.

As used herein, connexin 43 refers to native connexin 43 expressed by the hepatic stellate cell in which proliferation is to be inhibited, or may include connexin 43 encoded by a vector delivered to the cell or protein delivered to the cell. Connexin 43 delivered to the cell by vector or as a protein may be the same connexin 43 as expressed by the cell or it may be a homologue from another species.

The Cx43 may be human Cx43, for example comprising, consisting essentially of or consisting of the following sequence [SEQ ID NO: 1]:

MGDWSALGKLLDKVQAYSTAGGKVWLSVLFIFRILLLGTAVESAWGDE
QSAFRCNTQQPGCENVCYDKSFPISHVRFWVLQIIFVSVPTLLYLAHV
FYVMRKEEKLNKKEEELKVAQTDGVNVDMHLKQIEIKKFKYGIEEHGK
VKMRGGLLRTYIISILFKSIFEVAFLLIQWYIYGFSLSAVYTCKRDPC
PHQVDCFLSRPTEKTIFIIFMLVVSLVSLALNIIELFYVFFKGVKDRV
KGKSDPYHATSGALSPAKDCGSQKYAYFNGCSSPTAPLSPMSPPGYKL
VTGDRNNSSCRNYNKQASEQNWANYSAEQNRMGQAGSTISNSHAQPFD
FPDDNQNSKKLAAGHELQPLAIVDQRPSSRASSRASSRPRPDDLEI

Alternatively, the Cx43 may be rat Cx43, for example comprising, consisting essentially of or consisting of the following sequence [SEQ ID NO: 2]:

MGDWSALGKLLDKVQAYSTAGGKVWLSVLFIFRILLLGTAVESAWGDEQSA
FRCNTQQPGCENVCYDKSFPISHVRFWVLQIIFVSVPTLLYLAHVFYVMR
KEEKLNKKEEELKVAQTDGVNVEMHLKQIEIKKFKYGIEEHGKVKMRGGL
LRTYIISILFKSVFEVAFLLIQWYIYGFSLSAVYTCKRDPCPHQVDCFLS
RPTEKTIFIIFMLVVSLVSLALNIIELFYVFFKGVKDRVKGRSDPYHATT
GPLSPSKDCGSPKYAYFNGCSSPTAPLSPMSPPGYKLVTGDRNNSSCRNY
NKQASEQNWANYSAEQNRMGQAGSTISNSHAQPFDFPDDNQNAKKVAAGH
ELQPLAIVDQRPSSRASSRASSRPRPDDLEI

As used herein, “consists essentially of” or “consisting essentially of” means that a protein sequence includes one, two, three, five, ten or more amino acids at one or both ends of the described protein sequence, or that a nucleic acid molecule includes one, two, three, five, ten or more nucleotides at one or both ends of the described nucleic acid sequence, but that the additional amino acids or nucleotides do not materially affect the activity of the protein or the nucleic acid.

Active Cx43 is Cx43 that is translated, folded, post-translationally modified and localized within the cell, and which possesses the biological function or activity or Cx43, including the HSC proliferation activity of Cx43. Under any context in which the Cx43 protein is not, or not properly, translated, folded, post-translationally modified or localized within the cell, even if the gene is transcribed, a proliferative block may ensue. Active connexin 43 refers to connexin 43 that has the capability to up-regulate proliferation of an HSC in which it is expressed; and includes connexin 43 that is not inhibitorily phorphorylated, for example connexin 43 that is not phosphorylated at Ser368 or at an analogous position in a species homologue. As will be appreciated, inhibitory phosphorylation refers to phosphorylation at a serine, tyrosine or threonine residue which results in down-regulation of the protein as compared to the activity of the protein when not phosphorylated at that position. Active Cx43 may also possess the ability to assemble into and function within connexons, including when the connexon is assembled into an intercellular gap junction.

Thus, reference to the hepatic stellate cell proliferation activity of connexin 43 is reference to the effect of active connexin 43 of inhibiting proliferation of a hepatic stellate cell, as a result of expression of connexin 43 and maintenance of expressed connexin 43 in an active (i.e. non-inhibited) state within the cell.

The term cell (including in the context of HSCs) as used herein refers to and includes a single cell, a plurality of cells or a population of cells where context permits, unless otherwise specified. Similarly, reference to cells also includes reference to a single cell where context permits, unless otherwise specified.

The hepatic stellate cell is any hepatic stellate cell, including a quiescent or activated HSC, and including an HSC involved in hepatic fibrosis or a hepatic fibrosis related disorder. The HSC may be an in vitro HSC, including primary or immortalised, and an ex vivo HSC explanted from a subject, or may be an endogenous HSC in an in vivo context, including in a human subject. The HSC may be a transgenic HSC, including in an in vivo context, for example an animal model comprising a transgenic HSC, or in an in vitro context.

The HSC may be, any HSC in which proliferation or division is desired to be inhibited, including an activated HSC involved in hepatic fibrosis or a hepatic fibrosis related disorder, including a disorder in which HSC proliferation has been up-regulated. As used herein, proliferation of a cell refers to the process of DNA replication, growth and division, which leads to an increase in the total number of cells. Proliferation or inhibition of proliferation may readily be determined, for example by cell count, including in comparision with a population of HSCs that have not had proliferation inhibited, or by detection of proliferation specific cell markers, for example the proliferation specific marker PCNA.

Inhibiting proliferation or inhibition of proliferation of a cell includes rendering the cell incapable of replicating DNA, growing or dividing, or incapable of properly replicating DNA, growing or dividing, or reducing or retarding DNA replication, cell growth or division, in addition to inducing cell death by apoptosis or other mechanisms of cell death. Inhibiting may be performed in vitro or in vivo.

Down-regulating or down-regulation of the HSC proliferation activity of Cx43 refers to any mechanism of disrupting; interrupting, reducing, limiting, blocking or preventing the ability of Cx43 to promote or up-regulating proliferation, replication or cell cycle progression, thereby resulting in an inhibition of proliferation. Down-regulation includes physical alteration of Cx43, for example by post-translational modification including inhibitory phosphorylation, or by loss or lack of necessary post-translational modification. Down-regulation also includes genetic modification, including substantially decreasing or blocking expression of Cx43, including by increasing expression of Snai1 or by blocking transcription or translation of Cx43. Substantially decreasing refers to levels of expression of Cx43 that are, for example, approximately 50%, 45%, 40%, 35%, 30%, 25%, 20%, 15%, 10%, 5% or 0% of the levels of expression of Cx43 that would occur in a cell in which the HSC proliferation activity has not been down-regulated. Such genetic modification includes a modification of a nucleic acid encoding Cx43, such that part, or all, of the open reading frame has been deleted, replaced or interrupted such that substantially less or no gene product, no stable gene product, or no functional gene product is expressed. Such genetic modification also includes modification of a nucleic acid encoding Cx43, such that part, or all, of the Cx43 gene regulatory region has been deleted, replaced, interrupted or inhibited, resulting in substantially less or no protein being expressed from the gene encoding Cx43. Such genetic modification further includes modifying the cell to transcribe an antisense RNA transcript that is complementary to at least a fragment of the mRNA molecule that is transcribed from the Cx43 gene, resulting in no translation, or reduced translation of the Cx43 transcript. Such genetic modification further includes modifying the cell to transcribe or express a small interfering RNA (siRNA) molecule that targets a Cx43 gene transcript, resulting in no translation, or reduced translation of the Cx43 transcript.

Directly down-regulating (and direct down-regulation) refers to affecting the levels of Cx43 or the activity of Cx43 by direct action rather than via another effector. That is, a molecule that directly down-regulates the HSC proliferation activity of Cx43 may interact directly with Cx43 or may interact directly with a nucleic acid encoding Cx43, such as the upstream regulatory region for the Cx43 gene or with the Cx43 transcript. Thus, contacting a cell with TGF-β1 does not constitute direct down-regulation, since TGF-β1 exerts its effect on the HSC proliferation activity of Cx43 through activation of a signal cascade pathway and does not itself interact with the regulatory region of the Cx43 gene, with the Cx43 transcript or Cx43 itself.

The HSC proliferation activity of Cx43 may be down-regulated by an agent that down-regulates the HSC proliferation activity of Cx43, meaning that the agent, once delivered into a cell is capable of down-regulating such activity. Down-regulating may comprise delivering the agent into the HSC in which proliferation is to be inhibited, including contacting the HSC with the agent so that the agent is taken up by the cell or enters into the cell.

The agent may be capable of being delivered internally to a cell, for example by active or passive transport into the cell, or by diffusion into the cell. For example, if the agent is a small molecule, it may be soluble in the cell membrane and thus able to permeate the cell. The agent may also be modified to include a transport tag that will facilitate its transport into a cell. Specific transport tags may be used in order to direct the agent to be taken up by specific target cells. For example, the agent may be modified to include a galactose residue to increase uptake of the agent by hepatocytes, as is described in U.S. Pat. No. 6,844,319, which is herein fully incorporated by reference. Alternatively, the agent may be included in a biomaterial which increases or induces uptake of the agent by the cell; for example, by encapsulating the agent in a micellar or liposome preparation. Micelle and liposome delivery of peptides and proteins to cells is known. Alternatively, techniques for delivering nucleic acid molecules to a cell may be used, including transfection techniques or transduction techniques where the nucleic acid is a viral vector, or delivery of naked DNA to a cell.

An agent that down-regulates the HSC proliferation activity of Cx43 may be an inhibitor of Cx43 HSC proliferation activity, for example, a compound that inhibits, interferes with, competes with or interrupts the HSC proliferation activity of Cx43, including a molecule that causes, stimulates or enhances inhibitory serine phosphorylation of Cx43.

The agent may be a small molecule, a peptide, a protein, an antibody or a functional fragment thereof, or a protein kinase that inhibitorily phosphorylates Cx43 including at Ser368 or an analogous amino acid, for example protein kinase C.

Alternatively, the agent may be a molecule that inhibits or interferes with transcription of a nucleic acid encoding Cx43, for example a transcriptional repressor protein, including transcription factor Snai1. The agent may be a molecule that inhibits or interferes with translation of an RNA transcript encoding Cx43, for example a DNA enzyme or siRNA that interferes with translation of the an RNA transcript encoding Cx43 or an antisense RNA molecule directed against the Cx43 transcript.

Alternatively, down-regulating may comprise delivering a nucleic acid molecule encoding the agent, into the HSC in which proliferation is to be inhibited, including contacting the HSC with the nucleic acid molecule so that the nucleic acid molecule is taken up by the cell or enters into the cell. Once inside the HSC, the nucleic acid molecule will be capable of expressing the agent, and thus should include the necessary regulatory components to effect expression of the agent within the HSC.

Thus, the nucleic acid molecule may encode an inhibitor of Cx43, for example encode a protein kinase that inhibitorily phosphorylates Cx43, or a transcriptional repressor protein that inhibits transcription of a gene encoding Cx43. The nucleic acid molecule may encode a molecule capable of inhibiting or interfering with translation of an RNA transcript encoding Cx43 such as a DNA enzyme, an antisense RNA or an siRNA molecule directed against a transcript encoding the Cx43 protein.

As stated above, it will be appreciated that any nucleic acid molecule encoding an inhibitor of transcription, translation of Cx43 or of Cx43 protein will include any necessary regulatory elements required to effect expression, including transcription of the inhibitor in the HSC in which the inhibitor is to be expressed and in which proliferation is to be inhibited. Molecular biology and cloning techniques for generating nucleic acid molecules including particular coding sequences are known, and such nucleic acid molecules may be readily generated using routine laboratory methods.

For example, Snai1 or a nucleic acid molecule encoding Snai1 may be delivered into the HSC. Snai1 is a zinc finger transcription factor that has been demonstrated, as indicated in the Examples below, to bind to a Snai1 consensus binding sequence in the promoter region upstream of the Cx43 coding region, and which has been shown to down-regulate transcription of the Cx43 gene.

Snai1 may comprise, consist essentially of or consist of the following amino acid sequence [SEQ ID NO: 3]:

MPRSFLVRKPSDPNRKPNYSELQDSNPEFTFQQPYDQAHLLAAIPPPEI
LNPTASLPMLIWDSVLAPQAQPIAWASLRLQESPRVAELTSLSDEDSGK
GSQPPSPPSPAPSSFSSTSVSSLEAEAYAAFPGLGQVPKQLAQLSEAKD
LQARKAFNCKYCNKEYLSLGALKMHIRSHTLPCVCGTCGKAFSRPWLLQ
GHVRTHTGEKPFSCPHCSRAFADRSNLRAHLQTHSDVKKYQCQACARTF
SRMSLLHKHQESGCSGCPR

Alternatively, Snai1 may comprise, consist essentially of or consist of the following amino acid sequence [SEQ ID NO: 4]:

MPRSFLVRKPSDPRRKPNYSELQDACVEFTFQQPYDQAHLLAAIPPP
EVLNPAASLPTLIWDSLLVPQVQPVAWATLPLRESPRAAELTSLSDED
SGKSSQPPSPPSPAPSSFSSTSASSLEAEAFIAFPGLGQLPKQLARLS
VAKDPQSRKAFNCKYCNKEYLSLGALKMHIRSHTLPCVCTTCGKAFSR
PWLLQGHVRTHTGEKPFSCSHCNRAFADRSNLRAHLQTHSDVKRYQCQ
ACARTFSRMSLLHKHQESGCSGGPR

Down-regulation using Snai1, other protein agents, or small molecule inhibitors may be achieved using standard methods for delivery of a protein or a small molecule into a cell, including using micellar or liposome encapsulation techniques.

A DNA enzyme that targets the transcript of a gene encoding Cx43 may be delivered into the HSC. A DNA enzyme is a magnesium-dependent catalytic nucleic acid composed of DNA that can selectively bind to an RNA substrate by Watson-Crick base-pairing and potentially cleave a phosphodiester bond of the backbone of the RNA substrate at any purine-pyrimidine junction (Santiago, F. S., et al., (1999) Nat Med 5: 1264-1269). A DNA enzyme is composed of two distinct functional domains: a 15-nucleotide catalytic core that carries out phosphodiester bond cleavage, and two hybridization arms flanking the catalytic core; the sequence identity of the arms can be tailored to achieve complementary base-pairing with target RNA substrates.

The DNA enzyme will therefore have complementary regions that can anneal with regions on the transcript of a Cx43 gene flanking a purine-pyrimidine junction such that the catalytic core of the DNA enzyme is able to cleave the transcript at the junction, rendering the transcript unable to be translated to produce a functional Cx43 protein. In certain embodiments, the DNA enzyme is designed to cleave the Cx43 transcript between the A and the U residues of the AUG start codon.

For example, the transcript for Cx43 may comprise the following sequence [SEQ ID NO: 5]:

GGCUUUUAGC GUGAGGAAAG UACCAAACAG CAGCGGAGUU
UUAAACUUUA AAUAGACAGG UCUGAGUGCC UGAACUUGCC
UUUUCAUUUU ACUUCAUCCU CCAAGGAGUU CAAUCACUUG
GCGUGACUUC ACUACUUUUA AGCAAAAGAG UGGUGCCCAG
GCAACAUGGG UGACUGGAGC GCCUUAGGCA AACUCCUUGA
CAAGGUUCAA GCCUACUCAA CUGCUGGAGG GAAGGUGUGG
CUGUCAGUAC UUUUCAUUUU CCGAAUCCUG CUGCUGGGGA
CAGCGGUUGA GUCAGCCUGG GGAGAUGAGC AGUCUGCCUU
UCGUUGUAAC ACUCAGCAAC CUGGUUGUGA AAAUGUCUGC
UAUGACAAGU CUUUCCCAAU CUCUCAUGUG CGCUUCUGGG
UCCUGCAGAU CAUAUUUGUG UCUGUACCCA CACUCUUGUA
CCUGGCUCAU GUGUUCUAUG UGAUGCGAAA GGAAGAGAAA
CUGAACAAGA AAGAGGAAGA ACUCAAGGUU GCCCAAACUG
AUGGUGUCAA UGUGGACAUG CACUUGAAGC AGAUUGAGAU
AAAGAAGUUC AAGUACGGUA UUGAAGAGCA UGGUAAGGUG
AAAAUGCGAG GGGGGUUGCU GCGAACCUAC AUCAUCAGUA
UCCUCUUCAA GUCUAUCUUU GAGGUGGCCU UCUUGCUGAU
CCAGUGGUAC AUCUAUGGAU UCAGCUUGAG UGCUGUUUAC
ACUUGCAAAA GAGAUCCCUG CCCACAUCAG GUGGACUGUU
UCCUCUCUCG CCCCACGGAG AAAACCAUCU UCAUCAUCUU
CAUGCUGGUG GUGUCCUUGG UGUCCCUGGC CUUGAAUAUC
AUUGAACUCU UCUAUGUUUU CUUCAAGGGC GUUAAGGAUC
GGGUUAAGGG AAAGAGCGAC CCUUACCAUG CGACCAGUGG
UGCGCUGAGC CCUGCCAAAG ACUGUGGGUC UCAAAAAUAU
GCUUAUUUCA AUGGCUGCUC CUCACCAACC GCUCCCCUCU
CGCCUAUGUC UCCUCCUGGG UACAAGCUGG UUACUGGCGA
CAGAAACAAU UCUUCUUGCC GCAAUUACAA CAAGCAAGCA
AGUGAGCAAA ACUGGGCUAA UUACAGUGCA GAACAAAAUC
GAAUGGGGCA GGCGGGAAGC ACCAUCUCUA ACUCCCAUGC
ACAGCCUUUU GAUUUCCCCG AUGAUAACCA GAAUUCUAAA
AAACUAGCUG CUGGACAUGA AUUACAGCCA CUAGCCAUUG
UGGACCAGCG ACCUUCAAGC AGAGCCAGCA GUCGUGCCAG
CAGCAGACCU CGGCCUGAUG ACCUGGAGAU CUAG

The DNA enzyme may be synthesized using standard techniques known in the art, for example, standard phosphoramidite chemical ligation methods may be used to synthesize the DNA molecule in the 3′ to 5′ direction on a solid support, including using an automated nucleic acid synthesizer. Alternatively, the DNA enzyme may be synthesized by transcribing a nucleic acid molecule encoding the DNA enzyme. The nucleic acid molecule may be contained within a DNA or RNA vector, for delivery into a cellular expression system, for example, a viral vector. Suitable viral vectors include vaccinia viral vectors and adenoviral vectors.

The down-regulation thus may be achieved by exposing the HSC to the DNA enzyme so that the DNA enzyme is taken up by the cell, and is able to target and cleave a Cx43 transcript in the cell, resulting in decreased or no expression of functional Cx43 protein in the cell. Exposure may include transfection techniques, as are known in the art, or by microinjection techniques in which the DNA is directly injected into the cell. Exposure may also include exposing the cell to the naked DNA enzyme, as cells may take up naked DNA in vivo. Alternatively, if the DNA enzyme is included in a nucleic acid vector, such as a viral vector, the cell may be infected with the viral vector.

Alternatively, an antisense RNA molecule or a small interfering RNA (siRNA) molecule that inhibits expression of nucleic acid encoding Cx43 may be delivered into the HSC.

The antisense RNA molecule will contain a sequence that is complementary to at least a fragment of an RNA transcript of a Cx43 gene, and which can bind to the Cx43 transcript, thereby reducing or preventing the expression of the Cx43 gene in vivo. The antisense RNA molecule should have a sufficient degree of complementarity to the target mRNA to avoid non-specific binding of the antisense molecule to non-target sequences under conditions in which specific binding is desired, such as under physiological conditions.

The siRNA molecule may be any double-stranded RNA molecule, including a self-complementary single-stranded molecule that can fold back on itself to form the double-stranded siRNA, which induces gene-specific RNA interference in a cell, leading to decreased or no expression of the Cx43 gene in vivo. An siRNA typically targets a 19-23 base nucleotide sequence in a target mRNA, as described in Elbashir, et al. (2001) EMBO J. 20: 6877-6888, the contents of which is incorporated herein by reference. Generally, the sequence of one strand of the siRNA will be complementary to a portion of the mRNA of the target transcript, here the Cx43 transcript. Guidelines for designing siRNAs are known in the art, or siRNA designed to hybridize to a specific target may be obtained commercially (Ambion, Qiagen). For example, siRNAs with a 3′ UU dinucleotide overhang are often more effective in inducing RNA interference (RNAi). siRNA molecules directed against particular sequences are commercially available, or may be designed and purchased commercially. As well, computer programs are available to design siRNA sequences (e.g. Invitrogen).

For example, the siRNA may comprise a sense strand having the sequence r(CAG UGC ACA UGU AAC UAA U)dTdT [SEQ ID NO: 6] and an antisense strand having the sequence r(AUU AGU UAC AUG UGC ACU G) dTdT [SEQ ID NO: 7] which siRNA is directed against the sequence AAC AGU GCA CAU GUA ACU AAU [SEQ ID NO: 8] in the transcript (Rn_Gja1_{—}1_HP siRNA, Qiagen, Germany). Alternatively, the siRNA may comprise a sense strand having the sequence r(GGU AAG CUU CCC UGG UCU A)dTdT [SEQ ID NO: 9] and an antisense strand having the sequence r(UAG ACC AGG GAA GCU UAC C) dTdT [SEQ ID NO: 10] which siRNA is directed against the sequence CAG GUA AGC UUC CCU GGU CUA [SEQ ID NO: 11] in the transcript (Rn_Gja1_{—}5_HP siRNA, Qiagen, Germany).

Thus, in order to effect the down-regulation, the cell may be exposed to the antisense RNA, a nucleotide encoding the antisense RNA, the siRNA or a nucleotide encoding the siRNA, for example a nucleic acid vector containing a nucleic acid molecule which allows for transcription of an antisense transcript or a single-stranded, self-complementary siRNA molecule capable of forming a double-stranded siRNA. Such an antisense molecule, siRNA molecule or vector may be synthesized using nucleic acid chemical synthesis methods and standard molecular biology cloning techniques as described above.

Thus, inhibition of proliferation of an HSC may be achieved by delivering the agent into the cell, which comprises exposing the cell to the agent, allowing for uptake of the agent by the cell, allowing the agent to interact with Cx43, or DNA or RNA encoding Cx43 so as to down-regulate the HSC proliferation activity of Cx43.

The method may be performed in the context of an HSC affected by hepatic fibrosis or a hepatic fibrosis related disorder, including an HSC that contributes to or is involved in causing hepatic fibrosis or a hepatic fibrosis related disorder or an HSC that is activated due to or during hepatic fibrosis or a hepatic fibrosis related disorder.

Thus, the HSC in which proliferation is to be inhibited may be an HSC in vivo in a subject in need of treatment of hepatic fibrosis or a hepatic fibrosis related disorder, including a mammal, including a human.

A hepatic fibrosis related disorder refers to any disease, disorder or condition which may cause, result in, or is associated with hepatic fibrosis, including a primary fibrosis or a secondary fibrosis. Such disorders include hepatic fibrosis including, for example, cirrhosis, hepatitis C infection, hepatitis B infection, steatohepatitis associated with alcohol or obesity, hemochromatosis, Wilson's disorder, primary biliary cirrhosis (PBC), non-alcoholic steatohepatitis (NASH) or hepatic cancer.

Thus, delivering the agent or a nucleic acid encoding the agent into the cell includes administering an effective amount of the agent to the subject. The term “effective amount” as used herein means an amount effective, at dosages and for periods of time necessary to achieve the desired result, for example, to inhibit proliferation of an HSC in the subject and/or to treat hepatic fibrosis or a hepatic fibrosis related disorder.

The agent or a nucleic acid encoding the agent may be administered to the subject using standard techniques known in the art. The agent or a nucleic acid encoding the agent may be administered systemically, or may be administered directly at the site at of an HSC. Administration to the site includes injection to the site, or surgical implantation, and may include the hydrodynamic delivery of the agent or a nucleic acid encoding the agent via the afferent and efferent vessels of the liver, such as for example, the portal vein, the hepatic vein, or the bile duct.

The concentration and amount of the agent or a nucleic acid encoding the agent to be administered will vary, depending on the hepatic fibrosis or hepatic fibrosis related disorder to be treated, the type of cell associated with the hepatic fibrosis or hepatic fibrosis related disorder, the type of molecule that is administered, the mode of administration, and the age and health of the subject.

To aid in administration, the agent that down-regulates the HSC proliferation activity of Cx43 or a nucleic acid encoding the agent may be formulated as an ingredient in a pharmaceutical composition.

Therefore, there is provided a pharmaceutical composition comprising an agent that down-regulates the HSC proliferation activity of Cx43 or a nucleic acid encoding such an agent, and optionally a pharmaceutically acceptable diluent. Such pharmaceutical compositions may be for use in treating hepatic fibrosis or a hepatic fibrosis related disorder.

The compositions may routinely contain pharmaceutically acceptable concentrations of salt, buffering agents, preservatives and various compatible carriers. For all forms of delivery, the agent that down-regulates the HSC proliferation activity of Cx43 or a nucleic acid encoding such an agent may be formulated in a physiological salt solution. Since polypeptides may be unstable upon administration, where the agent that down-regulates the HSC proliferation activity of Cx43 is a polypeptide or a protein, it may be desirable to include the peptide or the protein in a liposome or other biomaterial useful for protecting and/or preserving the peptide or protein until it is delivered to the target cell.

The proportion and identity of the pharmaceutically acceptable diluent is determined by chosen route of administration, compatibility with live cells, and standard pharmaceutical practice. Generally, the pharmaceutical composition will be formulated with components that will not kill or significantly impair the biological properties of the agent that down-regulates the HSC proliferation activity of Cx43 or a nucleic acid encoding such an agent.

The pharmaceutical composition can be prepared by known methods for the preparation of pharmaceutically acceptable compositions suitable for administration to subjects, such that an effective quantity of the agent that down-regulates the HSC proliferation activity of Cx43 or a nucleic acid encoding such an agent, and any additional active substance or substances, is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the pharmaceutical compositions include, albeit not exclusively, solutions of the agent that down-regulates the HSC proliferation activity of Cx43 or a nucleic acid encoding such an agent, in association with one or more pharmaceutically acceptable vehicles or diluents, and contained in buffer solutions with a suitable pH and iso-osmotic with physiological fluids.

The pharmaceutical composition may be administered to a subject in a variety of forms depending on the selected route of administration, as will be understood by those skilled in the art. The composition of the invention may be administered topically, surgically or by injection to the desired site.

Solutions of the agent that down-regulates the HSC proliferation activity of Cx43 or a nucleic acid encoding such an agent may be prepared in a physiologically suitable buffer. Under ordinary conditions of storage and use, these preparations contain a preservative to prevent the growth of microorganisms, and that will maintain the function of the agent that down-regulates the HSC proliferation activity of Cx43. A person skilled in the art would know how to prepare suitable formulations. Conventional procedures and ingredients for the selection and preparation of suitable formulations are described, for example, in Remington's Pharmaceutical Sciences and in The United States Pharmacopeia: The National Formulary (USP 24 NF19) published in 1999.

The dose of the pharmaceutical composition that is to be used depends on the particular condition being treated, the severity of the condition, the individual subject parameters including age, physical condition, size and weight, the duration of the treatment, the nature of concurrent therapy (if any), the specific route of administration and other similar factors that are within the knowledge and expertise of the health practitioner. These factors are known to those of skill in the art and can be addressed with minimal routine experimentation.

Also contemplated are uses of an agent that down-regulates the HSC proliferation activity of Cx43 or a nucleic acid encoding such an agent, including use in the manufacture of a medicament, for inhibiting proliferation of an HSC or for treating hepatic fibrosis or a hepatic fibrosis related disorder, including various uses in accordance with the methods as described herein.

The present methods and uses are further exemplified by way of the following non-limited examples.

### EXAMPLES

### Materials and Methods

Cell culture conditions: Primary hepatic stellate cells were isolated from male Wistar rats according to a previously published procedure (Weiskirchen and Gressner, 2005). The protocol was approved by the Institutional Animal Care and Use Committee (IACUC) of the Biomedical Research Council of Singapore. The purity of primary HSCs was assessed by vitamin A autofluorescence one day after isolation. The cell line HSC-2 was described elsewhere (Maubach et al., 2008). All cells were cultivated in a humidified 37° C. incubator circulated with 5% CO₂. High glucose Dulbecco's modified Eagle medium (D-MEM) containing 10% fetal bovine serum, 100 units/ml penicillin and 100 μg/ml streptomycin was used during cell culture. Trypsin-EDTA was purchased from Biochrome (Germany). All other cell culture reagents were from Invitrogen (CA, USA).

Hepatic stellate cells treatment with recombinant human TGF-β1: Twenty four hours prior to treatment, HSCs were seeded in 75 cm² tissue culture flasks. At 60-70% cell confluence, recombinant human TGF-β1 (hTGF-[3]) was added at a final concentration of 1 or 10 ng/ml and incubated for 2, 6, 10, 24 or 30 hours. For the control treatment, only phosphate-buffered saline (PBS) was given to the cells. In some experiments, HSC-2 cells were treated with bisindolylmaleimide I (BIM I) at a final concentration of 5 μM for 30 min before treatment with 10 ng/ml hTGF-β1.

Reverse transcription and quantitative PCR: Total RNA was isolated from cells according to the manufacturer's protocol (RNA II kit, Machery-Nagel, Germany). All reagents for reverse transcription and real-time PCR were from Applied Biosystems (CA, USA). One microgram of total RNA was reverse transcribed to cDNA in a total reaction volume of 50 μl at conditions described in the RT kit (N8080234). Real-time PCR reactions were performed using the Fast Real Time PCR System (Applied Biosystems). Three microlitres of cDNA were used in a PCR reaction volume of 10 μl. The Taqman probes for target genes Cx43 and Snai1, as well as for endogenous control β-actin were Rn01433957_m1, Rn00441533_g1 and 4352341E, respectively. The PCR conditions were 95° C. for 20 s and 40 cycles of amplification at 95° C. for 3 s and 60° C. for 30 s.

SDS-PAGE and Western blot: Cell lysis and subsequent separation of total protein in SDS-PAGE followed by Western blot was performed as recently described (Lim et al., 2008). The membrane was blocked with 5% non-fat milk in TBS-Tween (TBS-T). The Cx43 (sc-9059, Santa Cruz Biotechnology, USA), phosphorylated Cx43 at serine 368 (pCx43 Ser368, 3511S, Cell Signaling Technology, USA), PCNA (Ab29, Abcam, UK) and β-actin (A2228, Sigma, USA) primary antibodies were applied at a dilution of 1:1000, 1:750, 1:5000 and 1:7500 respectively in blocking solution. After three washes in TBS-T, the appropriate secondary antibody conjugated with horse radish peroxidase (Santa Cruz Biotechnology, USA) was added at a dilution of 1:2000 in blocking solution. After three washes in TBS-T, the membrane was developed with ECL Plus (RPN2132, GE Healthcare, UK). The incubation with pCx43 antibody was performed overnight at 4° C. All other incubations were carried out for 1 hour at room temperature. Semi-quantitative densitometric analysis of Western blots was performed using the ImageJ software (W. Rasband, NIH; rsb.info.nih.gov/ij/).

Immunofluorescence staining: The immunofluorescence staining of HSC-2 cells was performed as described elsewhere (Maubach et al., 2007) using Cx43 (C-6219, Sigma, USA) and phosphorylated Cx43 at serine 368 (pCx43 Ser368, 3511S, Cell Signaling Technology, USA) antibodies at a dilution of 1:50. The secondary antibodies anti-rabbit Alexa488 and anti-rabbit Alexa555 (Invitrogen, USA) were used at a dilution of 1:200. Images were taken using the LEICA RMB-DM epifluorescence microscope (LEICA, Germany).

Analysis of gap junction intercellular communication: HSC-2 cells were grown in a 60 mm cell culture dish overnight. At 90% cell confluence, hTGF-β1 (final concentration 10 ng/ml) or carbenoxolone (final concentration 40 μM) was added and incubated for 6 hours. As control, only PBS was given to the cells. Alternatively, BIM I (final concentration 5 μM) was added 30 minutes before the addition of hTGF-[3]. After a brief rinse in PBS, cells were incubated in D-MEM without phenol red, containing 5,6-carboxyfluorescein diacetate (Research Organics, USA) at a final concentration of 50 μg/ml and incubated in a 37° C. humidified incubator for 30 min. The cells were then rinsed twice with PBS and D-MEM without phenol red was added before proceeding with the Fluorescence Recovery After Photobleaching (FRAP) assay. The FRAP application included in the software package of the LEICA TCS SP2 equipped with the DM6000 (LEICA, Germany) was used. A 63× immersed objective (Leica HCX APO L U-V-I 63×/0.90 Water UV) was used. The argon laser at 488 nm was used for excitation and the fluorescence signal was captured between 500 and 535 nm. The conditions were as follows: 5 pre-bleach scans at 10% laser power, 40 bleach scans at 100% laser power followed by 60 post-bleach scans at 15 seconds intervals. During the bleaching period, the Zoom mode was used to bleach a single cell (target cell) defined in a region of interest (ROI). All data were corrected for photobleaching during post-bleach acquisition using the whole scanned area. The time constant of recovery, tau (τ), was estimated by fitting the corrected experimental data (OriginPro 7 SR4, OriginLab USA) to the following function: F(t)=F₀+(F_{∞}−F₀)(1−e^{-t/τ}), with F(t) being the corrected fluorescence intensity and F_{∞} being the asymptotic value of the fluorescence intensity. The transfer constant (k) was calculated from k=1/τ and normalized by dividing by the number of cells in contact with the target cell. The fluorescence recovery for each cell was about 50%.

Electrophoretic mobility shift assay: Based on the rat Cx43 gene (NW_{—}001084790), biotinylated double-stranded oligonucleotide probe TGCTCAACCCAGTCAGGTGATGCCTGAACAAA-3 [SEQ ID NO: 12], with the Snai1 consensus sequence (underlined), was synthesized (Research Biolabs, Singapore). In the mutated double-stranded oligonucleotide, the Snai1 consensus sequence was changed to CAGGAA. Nuclear protein extract was obtained using the NE-PER Nuclear and Cytoplasmic Extraction kit (Pierce, USA). The electrophoretic mobility shift assay was performed using reagents from the Snai1 kit according to its protocol (AY1398, Panomics, USA). Briefly, 5 μg nuclear protein extract was incubated in a reaction mixture consisting of poly d(1-C), 5× binding buffer and nuclease-free water for 5 min before addition of 1 μl probe (stock 20 nM). The total reaction volume was 10 μl. For competition assay, 2 μl unlabeled probe (stock 2 μM) was added 5 min prior to the addition of labeled probe. The reaction was incubated at 15° C. for 30 min. The samples were separated in a 6% non-denaturing polyacrylamide gel (Invitrogen) and transferred onto a nylon membrane.

Snai1 and connexin 43 siRNAs transfection: Shortly before transfection, 1−2×10⁶ HSC-2 cells were seeded in 100 mm cell culture dishes and incubated at 37° C. The siRNA was added at a final concentration of 10 nM to 1 ml of D-MEM without serum, followed by 120 μl of HiPerfect transfection reagent (Qiagen, Germany) and incubated for 10 min. The siRNA/transfection reagent solution was added drop-wise to the cells and incubated for 24 or 48 hours. As mock control, only HiPerfect reagent was added to the cells. The Snai1 siRNAs used were Rn_Snai1_{—}1 and Rn_Snai1_{—}3 and the Cx43 siRNAs used were Rn_Gja1_{—}1 and Rn_Gja1_{—}5 (Qiagen, Germany).

Cell counting: After treatment, cells were washed once with PBS and detached using trypsin/EDTA. Following centrifugation at 800 rpm for 4 min, the cell pellet was resuspended in 1 ml D-MEM and the cells were counted using the forward scatter function of the GUAVA PCA-96 (Guava Technologies, CA, USA).

Statistical analysis: All quantitative results were presented as mean±SD. Experimental data were analyzed using two-tailed Student's t-test assuming equal variances and One-Way ANOVA with Scheffé's Post-Hoc test where applicable. The criterion for data significance is a p-value<0.05. The p-values presented in the figure legends are based on the Student's t-test, unless otherwise stated.

Results

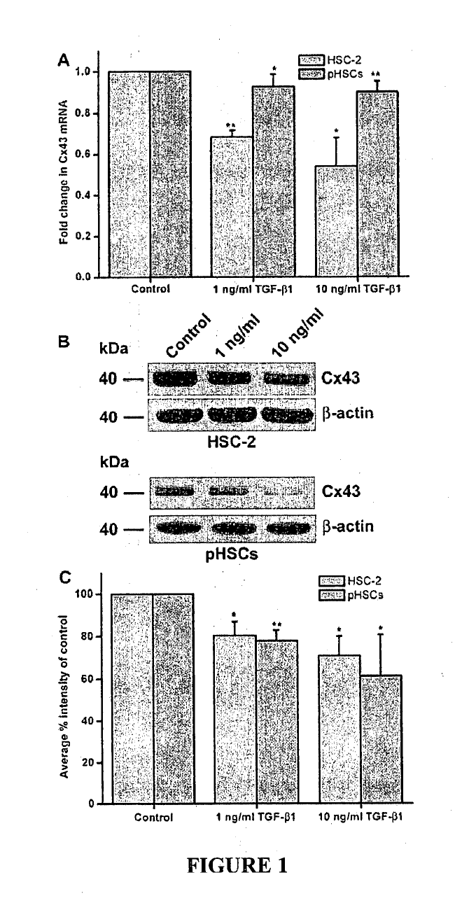

hTGF-β1 down-regulates Cx43 transcript and protein expression: To examine the regulation of Cx43 mRNA, HSC-2 cells were stimulated with pro-fibrogenic hTGF-β1 for 10 hours. Real-time PCR data showed that 1 ng/ml and 10 ng/ml hTGF-β1 led to a 30% and 45% decrease of Cx43 transcripts, respectively (FIG. 1A). In addition, it was also observed that hTGF-β1 down-regulated Cx43 protein (FIGS. 1B and C). Similar trends in Cx43 mRNA and protein regulation were observed when 10 days in vitro activated primary HSCs were subjected to hTGF-β1 treatment (FIGS. 1A, B and C).

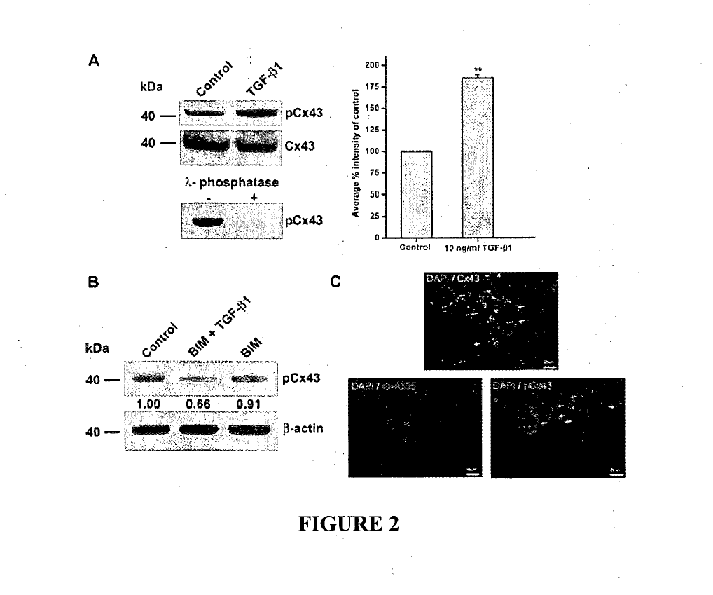

hTGF-β1 increases the phosphorylation of Cx43: After hTGF-β1 supplement, an increase in the phosphorylation of Cx43 at serine 368 (FIG. 2A) was observed, which is attributed to an increase in the proportion of pCx43 S368 in the total (decreasing) pool of Cx43. The authenticity of the pCx43 band was validated by its disappearance after λ-phosphatase treatment (FIG. 2A). Pre-treatment of the cells with the protein kinase C (PKC) inhibitor BIM I followed by hTGF-β1 reduces the phosphorylation of Cx43 at serine 368 (FIG. 2B).

Immunofluorescence staining was also performed for Cx43 and pCx43 S368 to study the cellular distribution of pCx43 S368 in HSC-2 cells. Cx43 is, to a great extent, distributed along the membrane whereas the pCx43 S368 shows a diffused or spotted staining in the cytoplasm with some membrane localization (FIG. 2C, arrows).

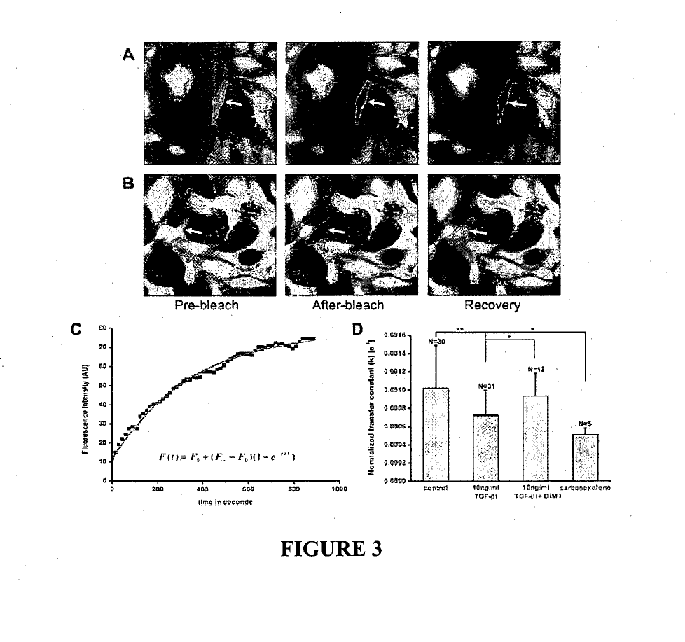

hTGF-β1 decreases gap junction intercellular communication between HSCs: Cx43 is the major gap junction protein expressed in the HSCs and has been shown to form functional gap junctions (Fischer et al., 2005). Here the gap-FRAP technique (Abbaci et al., 2007) was used to analyze the GJIC between HSCs. In order to validate this method, it was demonstrated that there is no spontaneous recovery of fluorescence in an isolated bleached cell (FIG. 3A, arrow), whereas a contacting cell recovers about 50% of its fluorescence (FIG. 3B, arrow). This indicates that the transfer of dye from an unbleached to a bleached cell via gap junctions is indeed being measured and not a recovery of the fluorescence signal as such. FIG. 3C is a representative graph, depicting the recovery function fitted to the experimental data. Carbenoxolone is an established GJIC inhibitor (Doll et al., 1968). In the present case, carbenoxolone reduced the dye transfer rate (k) to almost 50% (FIG. 3D). This result serves as a positive control for the reliability of the gap-FRAP technique to measure changes in GJIC. These findings showed that the transfer rate of the fluorescence dye 5,6-carboxyfluorescein diacetate was significantly lower in hTGF-β1-treated HSCs (FIG. 3D), implying reduced GJIC in these cells in comparison to PBS-treated HSCs (control). The TGF-β1-induced down-regulation of GJIC was found to be attenuated when the cells were treated with BIM I, a PKC inhibitor, prior to the addition of hTGF-β1 (FIG. 3D).

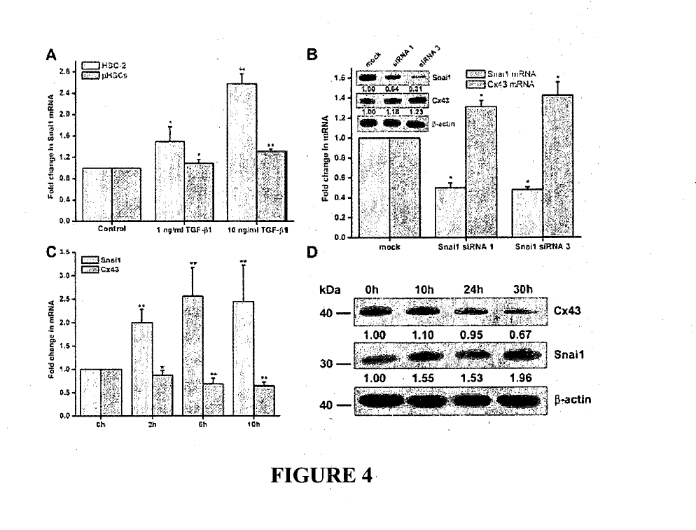

TGF-β1 down-regulates Cx43 expression via Snai1: TGF-β1 is known to up-regulate the expression of Snai1, a zinc finger transcription factor involved in epithelial-mesenchymal transition (EMT) (Peinado et al., 2003). Snai1, on the other hand, is necessary for the repression of the transcription of E-cadherin in epithelial tumor cells and Cx43 during EMT (Bathe et al., 2000; de Boer et al., 2007). FIG. 4A showed that hTGF-β1 induced the Snai1 mRNA in cell line HSC-2 and in in vitro activated primary HSCs. Transfection of HSC-2 with two Snai1-specific siRNAs (1 and 3) led to a down-regulation of Snai1 mRNA and protein by almost 50 percent (FIG. 4B). Concurrently, it was observed that the Cx43 was up-regulated on the mRNA (31% and 43%) and protein (18% and 23%) level following Snai1 siRNA 1 and 3 transfection, respectively (FIG. 4B). In order to further support the proposition that the regulation of Cx43 could in part be mediated by Snai1, an increase in Snai1 was demonstrated, which corresponds to a decrease in Cx43, on both the transcript and protein expression after hTGF-β1 treatment up to 30 hours (FIGS. 4, C and D).

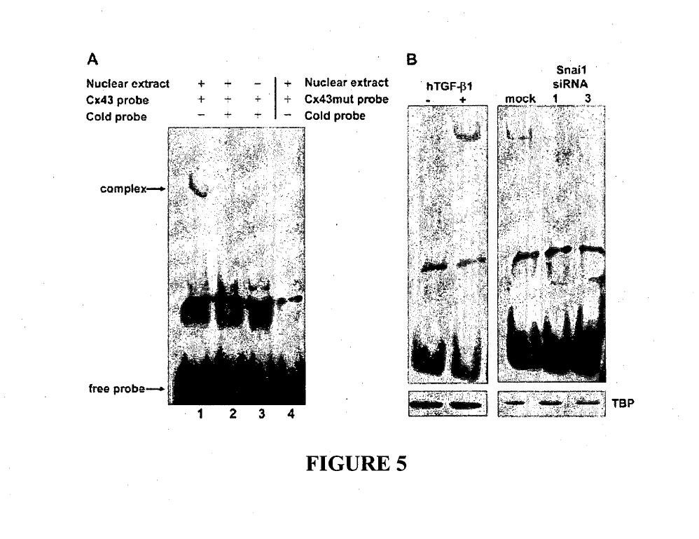

Nuclear extracts of HSCs bind to the Snai1 consensus sequence in the Cx43 promoter: To further assess the possibility that Snai1 has the potential to regulate Cx43 gene expression, electrophoretic mobility shift assay was performed using a biotinylated oligonucleotide probe based on the rat Cx43 promoter containing the Snai1 consensus sequence (CAGGTG) and nuclear extract from 12 days in vitro activated HSCs. This consensus sequence is situated 1412 by up-stream of the transcription initiation site. The binding of Snai1 to its consensus sequence was visualized by a mobility shift of the oligonucleotide probe in a 6% polyacrylamide gel (FIG. 5A, lane 1). This binding could be competed away by 200-fold excess of cold (unlabeled) probe (FIG. 5A, lane 2). In addition, no signal was detected in the absence of the nuclear extract (FIG. 5A, lane 3), and when a mutated biotinylated probe (CAGGAA) was used, where there is, a two base pair mutation in the Snai1 consensus sequence (FIG. 5A, lane 4), indicating that the binding observed was specific between the Snai1 proteins in the nuclear extract and the probe. Similar results were also obtained with the nuclear extract of the cell line HSC-2 (data not shown). Likewise, using the nuclear extract of HSC-2 treated with 10 ng/ml hTGF-β1 resulted in a more intense band, while cells transfected with Snai1 siRNAs produced weaker bands (FIG. 5B), further exemplifying the specificity of the binding between Snai1 and its consensus sequence in the Cx43 promoter.

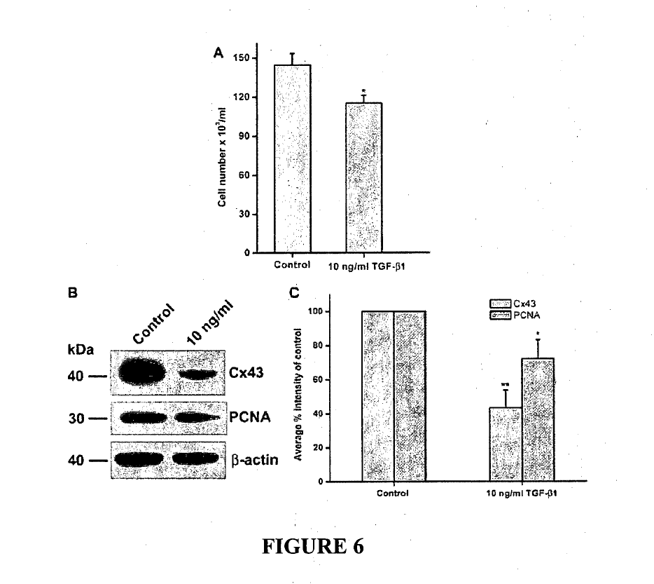

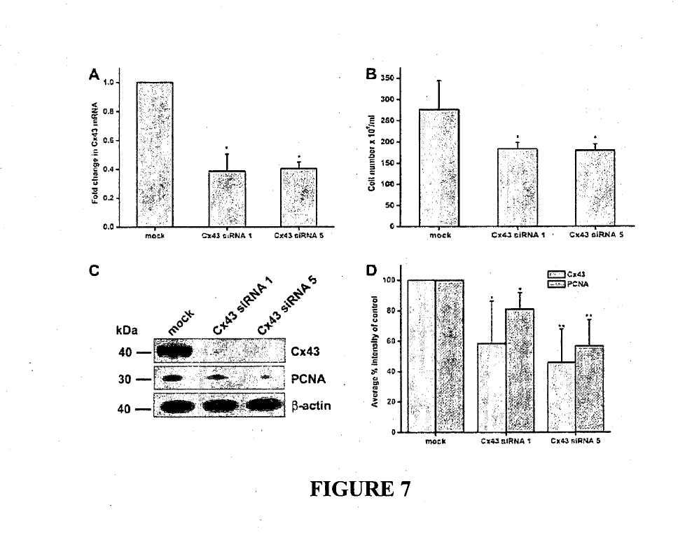

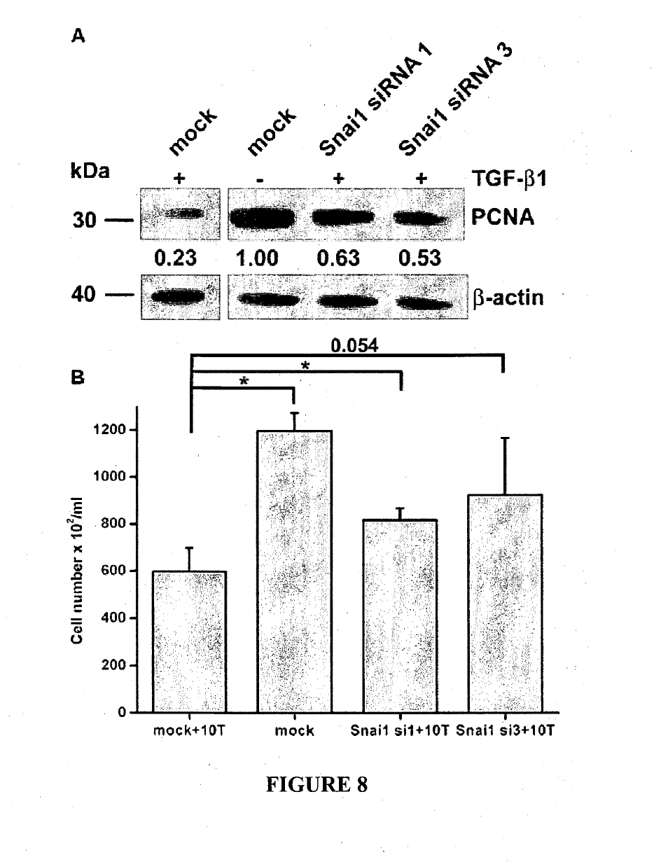

Connexin 43 regulates HSC proliferation: TGF-β1 is known to regulate the proliferation of cells. The effect on the proliferation of HSC-2 was demonstrated using cell count and immunoblot analysis of the proliferation marker, proliferating cell nuclear antigen (PCNA). Treatment of HSC-2 with hTGF-β1 led to a significant reduction in the cell number (FIG. 6A), as well as in the expression of PCNA and Cx43 (FIGS. 6, B and C). Apart from GJIC, the relevance of Cx43 in the TGF-β1-dependent regulation of HSC proliferation was investigated by using Cx43 siRNA to attenuate Cx43 mRNA level. Transfection of Cx43-specific siRNA 1 and 5 into HSC-2 caused the down-regulation of Cx43 mRNA by about 65% in both cases, demonstrating the efficacy of the siRNAs (FIG. 7A). Cell counting was performed after 48 hours treatment with Cx43 siRNA 1 and 5 to assess the cell proliferation. FIG. 7B illustrates clearly a significant decline in the total number of cells after transfection with Cx43 siRNAs in comparison to mock-transfected cells. Similarly, Cx43 siRNAs also led to a reduction in the expression of Cx43 protein (FIGS. 7, C and D), justifying the assumption that the Cx43 protein could be responsible for this decline. Furthermore, lower expression of PCNA in Cx43 siRNAs-transfected cells occurred than in mock-transfected cells (FIGS. 7, C and D). It was also found that the TGF-β1-down regulation of cell proliferation was attenuated by transfecting HSC-2 cells with Snai1 siRNA (FIGS. 8, A and B).

### SUMMARY

The data reveals that exogenous hTGF-β1 reduces Cx43 transcript and protein in a HSC cell line and in in vitro activated primary HSCs (FIG. 1). Additionally, there is an increase in the phosphorylation of Cx43 (Ser368) in the hTGF-β1-treated cells (FIG. 2A). Results indicate that the PKC is responsible for the phosphorylation of Cx43 at serine 368 (FIGS. 2B and 3D). This observation is consistent with earlier findings on serine 368 phosphorylation in Cx43 by PKC (Lampe et al., 2000). The cytosolic and partial membrane distribution of pCx43 (S368) shown by immunofluorescence (FIG. 2C) also suggests that the phosphorylation can affect not only the channel gating (Lampe et al., 2000), but also the trafficking and assembly into connexons. (Solan and Lampe, 2005). Taken together, the consequence is a lowered GJIC among the hTGF-β1-treated HSCs in comparison to control, as shown by gap-FRAP experiments (FIG. 3D). In other words, regulation of Cx43 by TGF-β1 appears to be bipartite, brought about by the short term (6 h) increase in pCx43 (Ser368) and the long term (24 h) down-regulation of Cx43 expression.

It was determined that TGF-β1 up-regulates Snai1 in HSC-2 and in in vitro activated primary HSCs (FIG. 4A). Use of Snai1 siRNAs led to a down-regulation of Snai1 and a simultaneous up-regulation of Cx43 (FIG. 4B). The EMSA results indicate that Snai1 can recognize specifically its binding site on the rat Cx43 promoter (FIG. 5A). This binding specificity is further supported by the inability of Snai1 to bind to the mutated consensus sequence. On the other hand, the down-regulation of Snai1 using siRNA diminished the binding (FIG. 5B). Furthermore, the results indicate that TGF-1:11 treatment leads not only to an increase in Snai1, but also to an increase in the binding of Snai1 to its consensus sequence (FIG. 5B).

TGF-β1 decreased the cell number (FIG. 6A), and the expression of PCNA (FIGS. 6, B and C), a marker for cell proliferation capability, of HSCs, which is coherent with the results of Shen and colleagues. Cx43 siRNAs were transfected into HSCs (FIG. 7A) and HSCs after Cx43 siRNA transfection proliferate slower than their mock-transfected counterparts as shown by a decrease in cell number (FIG. 7B) and PCNA expression (FIGS. 7, C and D), implying that TGF-β1 may mediate its effect on HSC proliferation to some degree through Cx43. TGF-β1 induced reduction in cell number and PCNA expression is attenuated by the suppression of Snai1 using Snai1 siRNA (FIG. 8).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

Abbaci, M., Barberi-Heyob, M., Stines, J. R., Blondel, W., Dumas, D., Guillemin, F. Didelon, J., 2007. Gap junctional intercellular communication capacity by gap-FRAP technique: a comparative study. Biotechnol J 2, 50-61.
Bataller, R., Brenner, D. A. 2001. Hepatic stellate cells as a target for the treatment of liver fibrosis. Semin Liver Dis. 21(3), 437.
Bataller, R., Brenner, D. A. 2005. Liver fibrosis. J. Clin Invest. 115(2), 209.
Baffle, E., Sancho, E., Franci, C., Dominguez, D., Monfar, M., Baulida, J. Garcia De Herreros, A., 2000. The transcription factor snail is a repressor of E-cadherin gene expression in epithelial tumour cells. Nat Cell Biol 2, 84-89.
Cassiman, D., Roskams, T., 2002. Beauty is in the eye of the beholder: emerging concepts and pitfalls inhepatic stellate cell research. J. Hepatol. 36(2), 200.
Dang, X., Doble, B. W. Kardami, E., 2003. The carboxy-tail of connexin-43 localizes to the nucleus and inhibits cell growth. Mol Cell Biochem 242, 35-38.
de Boer, T. P., van Veen, T. A., Bierhuizen, M. F., Kok, B., Rook, M. B., Boonen, K. J., Vos, M. A., Doevendans, P. A., de Bakker, J. M. van der Heyden, M. A., 2007. Connexin43 repression following epithelium-to-mesenchyme transition in embryonal carcinoma cells requires Snail1 transcription factor. Differentiation 75, 208-218.
De Craene, B., van Roy, F. Berx, G., 2005. Unraveling signalling cascades for the Snai1 family of transcription factors. Cell Signal 17, 535-547.
De Maio, A., Vega, V. L. Contreras, J. E., 2002. Gap junctions, homeostasis, and injury. J Cell Physiol 191, 269-282.
De Minicis, S., Seki, E., Uchinami, H., Kluwe, J., Zhang, Y., Brenner, D. A. Schwabe, R. F., 2007. Gene expression profiles during hepatic stellate cell activation in culture and in vivo. Gastroenterology 132, 1937-1946.
Doll, R., Langman, M. J. Shawdon, H. H., 1968. Treatment of gastric ulcer with carbenoxolone: antagonistic effect of spironolactone. Gut 9, 42-45.
Eyre, T. A., Ducluzeau, F., Sneddon, T. P., Povey, S., Bruford, E. A. Lush, M. J., 2006. The HUGO Gene Nomenclature Database, 2006 updates. Nucleic Acids Res 34, D319-321.
Fischer, R., Reinehr, R., Lu, T. P., Schonicke, A., Warskulat, U., Dienes, H. P. Haussinger, D., 2005. Intercellular communication via gap junctions in activated rat hepatic stellate cells. Gastroenterology 128, 433-448.
Friedman, S. L., 2003. Liver fibrosis—from bench to bedside. J. Hepatol. 38 Suppl 1, S38.
Friedman, S. L., 2008. Hepatic stellate cells: protean, multifunctional, and enigmatic cells of the liver. Physiol Rev 88, 125-172.
Gonzalez, H. E., Eugenin, E. A., Garces, G., Solis, N., Pizarro, M., Accatino, L. Saez, J. C., 2002. Regulation of hepatic connexins in cholestasis: possible involvement of Kupffer cells and inflammatory mediators. Am J Physiol Gastrointest Liver Physiol 282, G991-G1001.
Goodenough, D. A., Goliger, J. A. Paul, D. L., 1996. Connexins, connexons, and intercellular communication. Annu Rev Biochem 65, 475-502.
Gramsch, B., Gabriel, H. D., Wiemann, M., Grummer, R., Winterhager, E., Bingmann, D. Schirrmacher, K., 2001. Enhancement of connexin 43 expression increases proliferation and differentiation of an osteoblast-like cell line. Exp Cell Res 264, 397-407.
Geerts, A., 2004. On the origien of stellate cells: mesodermal, endodermal or neuro-ectodermal? J. Hepatol. 40(2), 331.
Gressner, O. A., Weiskirchen, R. Gressner, A. M., 2007. Evolving concepts of liver fibrogenesis provide new diagnostic and therapeutic options. Comp Hepatol 6, 7.
Hellerbrand, C., Stefanovic, B., Giordano, F., Burchardt, E. R. Brenner, D. A., 1999. The role of TGFbetal in initiating hepatic stellate cell activation in vivo. J Hepatol 30, 77-87.
Jia, G., Cheng, G., Gangahar, D. M. Agrawal, D. K., 2008. Involvement of connexin 43 in angiotensin II-induced migration and proliferation of saphenous vein smooth muscle cells, via the MAPK-AP-1 signaling pathway. J Mol Cell Cardiol 44, 882-890.
Jiang, F., Parsons, C. J. Stefanovic, B., 2006. Gene expression profile of quiescent and activated rat hepatic stellate cells implicates Wnt signaling pathway in activation. J Hepatol 45, 401-409.
Kaimori, A., Potter, J., Kaimori, J. Y., Wang, C., Mezey, E. Koteish, A., 2007. Transforming growth factor-beta1 induces an epithelial-to-mesenchymal transition state in mouse hepatocytes in vitro. J. Biol. Chem. 282, 22089-22101.
Kanzler, S., Lohse, A. W., Keil, A., Henninger, J., Dienes, H. P., Schirmacher, P., Rose-John, S., zum Buschenfelde, K. H. Blessing, M., 1999. TGF-beta1 in liver fibrosis: an inducible transgenic mouse model to study liver fibrogenesis. Am J Physiol 276, G1059-1068.
Kato, J., Ido, A., Hasuike, S., Uto, H., Hori, T., Hayashi, K., Murakami, S., Terano, A. Tsubouchi, H., 2004. Transforming growth factor-beta-induced stimulation of formation of collagen fiber network and anti-fibrotic effect of taurine in an in vitro model of hepatic fibrosis. Hepatol Res 30, 34-41.
Kharbanda, K. K., Rogers, D. D., 2nd, Wyatt, T. A., Sorrell, M. F. Tuma, D. J., 2004. Transforming growth factor-beta induces contraction of activated hepatic stellate cells. J Hepatol 41, 60-66.
Lampe, P. D., TenBroek, E. M., Burt, J. M., Kurata, W. E., Johnson, R. G. Lau, A. F., 2000. Phosphorylation of connexin43 on serine-368 by protein kinase C regulates gap junctional communication. J Cell Biol 149, 1503-1512.
Lim, M. C., Maubach, G. Zhuo, L., 2008. Glial fibrillary acidic protein splice variants in hepatic stellate cells—expression and regulation. Mol Cells 25, 376-384.
Loewenstein, W. R., 1981. Junctional intercellular communication: the cell-to-cell membrane channel. Physiol Rev 61, 829-913.
Maubach, G., Lim, M. C., Kumar, S. Zhuo, L., 2007. Expression and upregulation of cathepsin S and other early molecules required for antigen presentation in activated hepatic stellate cells upon IFN-gamma treatment. Biochim Biophys Acta 1773, 219-231.
Maubach, G., Lim, M. C. Zhuo, L., 2008. Nuclear cathepsin F regulates activation markers in rat hepatic stellate cells. Mol Biol Cell 19, 4238-4248.
Moorby, C. Patel, M., 2001. Dual functions for connexins: Cx43 regulates growth independently of gap junction formation. Exp Cell Res 271, 238-248.
Neuhaus, J., Heinrich, M., Schwalenberg, T. Stolzenburg, J. U., 2008. TGF-beta1 Inhibits Cx43 Expression and Formation of Functional Syncytia in Cultured Smooth Muscle Cells from Human Detrusor. Eur Urol.
Peinado, H., Quintanilla, M. Cano, A., 2003. Transforming growth factor beta-1 induces snail transcription factor in epithelial cell lines: mechanisms for epithelial mesenchymal transitions. J Biol Chem 278, 21113-21123.
Pimentel, R. C., Yamada, K. A., Kleber, A. G. Saffitz, J. E., 2002. Autocrine regulation of myocyte Cx43 expression by VEGF. Circ Res 90, 671-677.
Saile, B., Matthes, N., Knittel, T. Ramadori, G., 1999. Transforming growth factor beta and tumor necrosis factor alpha inhibit both apoptosis and proliferation of activated rat hepatic stellate cells. Hepatology 30, 196-202.
Schug, J. 2003. Using TESS to Predict Transcription Factor Binding Sites in DNA Sequence, In: Baxevanis, A. D. (Eds.), Current Protocols in Bioinformatics. J. Wiley and Sons, pp.
Segretain, D. Falk, M. M., 2004. Regulation of connexin biosynthesis, assembly, gap junction formation, and removal. Biochim Biophys Acta 1662, 3-21.
Shen, H., Huang, G. J. Gong, Y. W., 2003. Effect of transforming growth factor beta and bone morphogenetic proteins on rat hepatic stellate cell proliferation and trans-differentiation. World J Gastroenterol 9, 784-787.
Solan, J. L. Lampe, P. D., 2005. Connexin phosphorylation as a regulatory event linked to gap junction channel assembly. Biochim Biophys Acta 1711, 154-163.
Verrecchia, F. Mauviel, A., 2007. Transforming growth factor-beta and fibrosis. World J Gastroenterol 13, 3056-3062.
Weiskirchen, R. Gressner, A. M., 2005. Isolation and culture of hepatic stellate cells. Methods Mol Med 117, 99-113.
Wyatt, L. E., Chung, C. Y., Carlsen, B., Iida-Klein, A., Rudkin, G. H., Ishida, K., Yamaguchi, D. T. Miller, T. A., 2001. Bone morphogenetic protein-2 (BMP-2) and transforming growth factor-beta1 (TGF-beta1) alter connexin 43 phosphorylation in MC3T3-E1 Cells. BMC Cell Biol 2, 14.

1. A method of inhibiting proliferation of a hepatic stellate cell, comprising:
directly down-regulating the hepatic stellate cell proliferation activity of connexin 43 in the hepatic stellate cell.

2. The method of claim 1, wherein the hepatic stellate cell is in vitro.

3. The method of claim 1, wherein the hepatic stellate cell is in vivo.

4. The method of claim 3, wherein the hepatic stellate cell is a hepatic stellate cell affected by hepatic fibrosis or a hepatic fibrosis related disorder.

5. The method of claim 1, wherein the hepatic stellate cell is an activated hepatic stellate cell.

6. The method of claim 1, wherein the down-regulating comprises delivering into the hepatic stellate cell Snai1 or a nucleic acid molecule encoding Snai1.

7. The method of claim 1, wherein the down-regulating comprises delivering into the hepatic stellate cell an siRNA directed against a Cx43 transcript or a nucleic acid molecule encoding an siRNA directed against a Cx43 transcript.

8. The method of claim 1, wherein the down-regulating comprises delivering into the hepatic stellate cell an antisense RNA directed against a Cx43 transcript or a nucleic acid molecule encoding an antisense RNA directed against a Cx43 transcript.

9. The method of claim 1, wherein the down-regulating comprises delivering into the hepatic stellate cell a DNA enzyme directed against a Cx43 transcript or a nucleic acid molecule encoding a DNA enzyme directed against a Cx43 transcript.

10-15. (canceled)

16. The method of claim 4, wherein the hepatic stellate cell is an activated hepatic stellate cell.

17. The method of claim 4, wherein the hepatic fibrosis related disorder is cirrhosis, hepatitis C infection, hepatitis B infection, steatohepatitis associated with alcohol or obesity, hemochromatosis, Wilson's disorder, primary biliary cirrhosis, non-alcoholic steatohepatitis or hepatic cancer.

18. The method of claim 3, wherein the agent is administered systemically to a subject.

19. The method of claim 3, wherein the agent is administered to a subject at the site of the hepatic stellate cell in which proliferation is to be inhibited.

20. The method of claim 7, wherein the siRNA is directed against a sequence comprising the sequence set forth in SEQ ID NO: 8.

21. The method of claim 7, wherein the siRNA is directed against a sequence comprising the sequence set forth in SEQ ID NO: 11.

22. The method of claim 6, wherein the Snai1 comprises the amino acid sequence set forth in SEQ ID NO: 3 or SEQ ID NO: 4.
